(19) United States
(12) Patent Application Publication
(10) Pub. No.: US 2016/0166818 A1
(43) Pub. Date: Jun. 16, 2016

(54) METHODS OF INTERCELLULAR AND INTRACELLULAR DELIVERY SUBSTANCES ENCAPSULATED IN A DELIVERY VEHICLE

(71) Applicant: EP Technologies LLC, Akron, OH (US)

(72) Inventors: Sameer Kalghatgi, Copley, OH (US); Tsung-Chan Tsai, Cuyahoga Falls, OH (US); Daphne Pappas Antonakas, Hudson, OH (US); Robert L. Gray, Kent, OH (US)

(21) Appl. No.: 14/967,512

(22) Filed: Dec. 14, 2015

Related U.S. Application Data
(60) Provisional application No. 62/091,126, filed on Dec. 12, 2014.

(51) Int. Cl. A61M 37/00; A61N 1/44

(52) U.S. Cl. CPC A61M 37/00; A61N 1/44; A61M 2037/0007

(57) ABSTRACT

Exemplary methods of improving the delivery of a substance encapsulated in a delivery vehicle to cells of interest in skin, tissue or tumor are disclosed herein.

### RELATED APPLICATIONS

This non-provisional utility patent application is based on and claims priority to U.S. Provisional Patent Application Ser. No. 62/091,126 titled, METHODS OF INTERCELLULAR AND INTRACELLULAR DELIVERY SUBSTANCES ENCAPSULATED IN A DELIVERY VEHICLE, which was filed on Dec. 12, 2014, and which is incorporated herein by reference in its entirety.

### TECHNICAL FIELD

The present disclosure relates generally to methods for improving delivery of substances encapsulated in a delivery vehicle to areas of interest between cells of skin, tissue or tumor and/or into cells of interest in skin, tissue or tumor. The substances may be drugs, cosmeceuticals, DNA, RNA, proteins, DNA vaccines, protein based vaccines or the like.

### BACKGROUND OF THE INVENTION

Targeted delivery of substances to areas between cells of skin, tissue or tumor and into cells of interest in skin, tissue or tumor remains challenging. Development of delivery vehicles has improved delivery of substances across cellular membranes, but a number of large issues remain.

Electroporation is one method for drug delivery that consists of applying high-voltage pulses to skin. The applied high-voltage plays a dual role. First, it creates new pathways for enhancing drug permeability and second, it provides an electrical force for driving like-charged molecules through the newly created pores. Electroporation is usually used on the unilamellar phospholipid bilayers of cell membranes. However, it has been demonstrated that electroporation of skin is feasible, even though the stratum corneum (SC) contains multilamellar, intercellular lipid bilayers with phospholipids and no living cells.

Electroporation of skin requires high transdermal voltages (˜100 V or more, usually >100 V). In transdermal electroporation, the predominant voltage drop of an applied electric pulse to the skin develops across the SC. This voltage distribution causes electric breakdown (electroporation) of the SC. If the voltage of the applied pulses exceeds a voltage threshold of about 75 to 100 V, micro channels or “local transport regions” are created through the breakdown sites of the SC.

DNA introduction is the most common use for electroporation. Electroporation of isolated cells has also been used for (1) introduction of enzymes, antibodies, and other biochemical reagents for intracellular assays; (2) selective biochemical loading of one size cell in the presence of many smaller cells; (3) introduction of virus and other particles; (4) cell killing under nontoxic conditions; and (5) insertion of membrane macromolecules into the cell membrane.

The presence of electrodes in contact with skin/tissue and the delivery of current into skin/tissue in this manner leads to patient discomfort, muscle contractions, pain and sometimes even skin damage or burns. In addition, electroporation often takes hours, e.g. 6 to 24 hours, to drive therapeutic amount of drugs or other molecules transdermally. Further, treatments over large area of skin, tissue or tumor are not feasible or safe using electroporation as patient discomfort, skin damage, muscle contractions, and pain due to flow of current over large areas would be extensive.

U.S. Pat. No. 8,455,228, entitled “Method to Facilitate Directed Delivery and Electroporation Using a Charged Steam”, state that “the method and apparatus in accordance with the present invention are effective in using an electrical field to adjust the electrochemical potential of a target molecule thereby providing molecular transport of the target molecule into and/or across the tissue by a diffusive transport mechanism.” The '228 patent discloses a first embodiment with dielectric properties to assure that it will hold a charge sufficient to polarize charged entities contained within a vessel and a plurality of electroporation applicators. The '228 patent disclosure suffers from several deficiencies. First, it requires molecules that may be polarized or charged, second it requires electroporation applicators and third, the molecule is contacted with plasma during the process, which may modify the molecular structure causing adverse results.

The '228 patent also discloses a second embodiment utilizing a plasma jet with a ground ring around an inner chamber. The disclosure related to this device containing cells suspended in fluid in the inner chamber and promoting uptake into the cells; or injecting plasmid intradermally and exposure of the injection site to plasma.

US patent publication No. 2014/0188071 discloses a method of applying a substance to the skin and applying plasma to the same area. The '071 publication disclose an open cell foam to hold a drugs, water etc. and applies plasma through the open cell foam. Applying plasma through the open cell foam and contacting the drugs with plasma may alter the molecular structure of the drugs and cause undesirable side effects and/or render the drug ineffective.

US patent publication 2012/0288934 discloses a plasma jet and the active substance is applied to the skin with the gas stream of the plasma jet and is transported onto the region of the living cells through the barrier door that has been opened by the plasma. Applying the active substance with the gas stream of the plasma jet may alter the molecular structure of the active substance and cause undesirable side effects and/or render the active substance ineffective.

### SUMMARY

Methods of improving delivery of substances encapsulated in a delivery vehicle to areas of interest between cells of skin, tissue or tumor and into cells of interest in skin, tissue or tumor are disclosed herein.

Exemplary methods include exposing skin, tissue or tumor to a plasma source and bringing a delivery vehicle encapsulating a substance into contact with cells of skin, tissue or tumor. In one aspect, the delivery vehicle is brought into contact with cells in skin, tissue or tumor after cells in skin, tissue or tumor have been exposed to a plasma source.

In another aspect, cells in skin, tissue or tumor are exposed to a plasma source after a delivery vehicle has been brought into contact with cells in skin, tissue or tumor.

In another aspect, delivery vehicles injected in to skin, tissue or tumor are delivered intracellularly in to cells after skin, tissue or tumor has been exposed to a plasma source

In yet another aspect, delivery vehicles injected in to skin, tissue or tumor release their contents in the vicinity of cells in skin, tissue or tumor when skin, tissue or tumor is exposed to a plasma source

### BRIEF DESCRIPTION OF THE DRAWINGS

These and other features and advantages of the present invention will become better understood with regard to the following description and accompanying drawings.

FIG. 1 is an exemplary illustration of applying plasma to skin, tissue or tumor;

FIG. 2 is an exemplary illustration of applying a delivery vehicle to porated skin, tissue or tumor;

FIG. 2A is an enlarged portion of FIG. 2;

FIG. 3 is an exemplary illustration of using plasma to release the substance contained in the delivery vehicle;

FIG. 4 is an exemplary enlarged illustration of the skin, tissue or tumor with the substance released from the delivery vehicle;

FIG. 5 is an exemplary illustration of applying plasma to skin, tissue or tumor to move the delivery vehicle into targeted cells in skin, tissue or tumor;

FIG. 6 is an enlarged exemplary illustration of the delivery vehicles located intercellularly and intracellularly;

FIG. 7 is an exemplary illustration of applying plasma to tissue having delivery vehicles located intercellularly and intracellularly to release the contents of the delivery vehicle;

FIG. 8 is an exemplary illustration of the delivery vehicles inside the cells wherein the contents of the delivery vehicle have been released;

FIG. 9 is an exemplary methodology of delivering a substance to targeted areas between cells;

FIG. 10 is another exemplary methodology of delivering a substance to targeted areas between cells;

FIG. 11 is another exemplary methodology of delivering a substance to targeted areas between cells;

FIG. 12 is another exemplary methodology of delivering a substance to targeted areas between cells and within cells;

FIG. 13 is another exemplary methodology of delivering a substance to targeted areas between cells and within cells;

FIG. 14 (A-B) provides graphs showing the depth of permeation and the amount of a delivery vehicle containing an encapsulated substance at different depths achieved by exposing cells to electroporation or a plasma source, as described in Example 1.

FIG. 15 (A-B) provides micrographs visualizing the detectable label on an delivery vehicle containing an encapsulated substance and present at various skin depths following electroporation or plasma source exposures, as described in Example 1;

FIG. 16 (A-B) provides graphs showing the depth of permeation of delivery vehicle versus the number of pulses at different applied plasma pulse durations;

FIG. 17 provides graphs showing the depth of permeation of delivery vehicle versus the applied plasma voltage;

FIG. 18 provides graphs showing the depth of permeation versus the pulse duration;

FIG. 19 provides graphs showing the depth of permeation of delivery vehicle encapsulating a substance versus frequency of plasma operation; and

FIGS. 20 and 21 provide graphs showing results of using plasma to cause the delivery vehicle located in the skin to release its encapsulated contents.

### DETAILED DESCRIPTION

Delivery vehicles for encapsulated substances have been developed to facilitate transport of substances across cellular membranes. Applicants have unexpectedly discovered, and describe herein, methods of using a plasma source in combination with delivery vehicle technology to further improve targeted delivery of substances encapsulated in delivery vehicles to areas between cells in skin, tissue or tumor and into cells of interest in skin, tissue or tumor. The methods can be carried out using non-thermal plasma sources, such as the plasma sources disclosed in U.S. Non-Provisional application Ser. No. 14/500144, filed on Sep. 29, 2014, which claims priority to U.S. Provisional Application Ser. No. 61/883701, filed Sep. 27, 2013, both of which are titled “Methods and Apparatus for Delivery of Molecules Across Layers of the Skin,” and both of which are incorporated herein by reference, in their entirety for the teachings therein. Tissue as used herein, refers to epithelial, mucosal, connective and muscle tissue in the body.

Exemplary methods include the steps of exposing skin, tissue, tumor or cells to a plasma source and bringing a delivery vehicle in contact with the skin, tissue, tumor or cells. Depending on the particular results desired, these steps can be carried out in any order. In addition, exemplary steps in one exemplary method may be included in other exemplary embodiments. Carrying out an exposure of the skin, tissue, tumor or cells to a non-thermal plasma source first improves the ability of delivery vehicles to penetrate surfaces, as described in Example 1. Carrying out the exposure of the skin, tissue, tumor or cells to a non-thermal plasma source after a delivery vehicle has been delivered to a desired area between cells or into the desired cells is expected to produce a directed release of the contents of the delivery vehicle. In some embodiments, a combination of plasma source exposures both prior to and after bringing a delivery vehicle into a desired area between cells or into cells is carried out to both, facilitate penetration of surfaces (e.g., skin, tissue, tumor, cells of interest, or any other cells), and to direct release of the contents or encapsulated substance contained in the delivery vehicle.

In certain embodiments, skin is first exposed to a plasma source under conditions, which make use of the plasma source to open (“plasmaporate”) the skin to enable the transport of molecules through the skin via the newly formed pores. In certain embodiments, the skin is already disturbed, e.g., due to a wound, presence of a tumor or other opening. Thus, in some embodiments, epidermal cells are first exposed to a plasma source under conditions which plasmaporate the epidermal cells. Any set of conditions appropriate to plasmaporate the skin and/or epidermal cells can be used. In some embodiments, the plasma source is applied at a voltage of about 3-30 kV, including about 4-10 kV, and about 11-20 kV, and about 21-30 kV for a pulse duration between about 1 nanosecond and about 1 millisecond, including about 1 nanosecond to about 500 nanoseconds, 1 microsecond to 10 microseconds, 10 microseconds to about 100 microseconds, and also including about 250 microseconds to about 750 microseconds. The delivery vehicle is then topically applied to the skin or epidermis, which has been opened to transport of molecules. In some embodiments, a permeation enhancer is further applied to the skin or epidermis to facilitate transport of the delivery vehicle through the skin or epidermis. The permeation enhancer can be any permeation enhancer known in the art that can be safely and effectively be used in the methods disclosed herein. In some embodiments, the permeation enhancer contains at least one of ethanol, glycerin, polyethylene glycol and isopropanol.

Once the delivery vehicle has been delivered to the cells or tissue of interest (about 1-60 minutes later), release of an encapsulated substance from the delivery vehicle can be carried out either through the breakdown of the delivery vehicle by the skin, tissue, tumor or cells in the body over time, or, in some embodiments, facilitated by exposing the skin, tissue, tumor or cells to which the delivery vehicle was topically applied to a plasma source under conditions which porate the delivery vehicle. Any set of conditions appropriate to porate the delivery vehicle can be used. In some embodiments, poration of the delivery vehicle is carried out by exposure to a plasma source at greater than about 30 kV over between about 1 and 500 nanoseconds. In some embodiments, delivery of the delivery vehicle to a subcellular location of interest within cells of interest in skin, tissue or tumor is facilitated by exposure to a plasma source under conditions which porate the cells of interest prior to porating the delivery vehicle. Any set of conditions appropriate to porate the cells of interest can be used. In some embodiments, the cells of interest are porated by exposure to a plasma source at between about 10 and 30 kV, including about 15-25 kV, over pulse duration of about 10 nanoseconds to about 1 microsecond, including about 100 nanoseconds to about 750 nanoseconds.

In certain embodiments, the delivery vehicle is brought into contact with tissue, tumor or cells by injecting the delivery vehicle inside the skin. In some embodiments, this is done prior to exposing cells to a plasma source under conditions which porate the delivery vehicle, such as those conditions described above. In some embodiments, further exposures to a plasma source are carried out before and/or after injecting the delivery vehicle inside the skin, tissue or tumor under conditions which porate the skin, tissue or tumor and/or porate cells inside the skin, tissue or tumor such as those conditions described above, prior to porating the delivery vehicle.

The delivery vehicle can be any delivery vehicle known in the art that can safely and effectively be used with the methods disclosed herein. Acceptable delivery vehicles for use in the methods described herein will be non-toxic, biocompatible, inert, non-functional, non-immunogenic, and biodegradable. In some embodiments, the delivery vehicle is a liposome, an artificial virosome, a bacterial phage like carrier, or a micelle. Other exemplary delivery vehicles include lipoprotein-based drug carriers, nanoparticle drug carriers, and dendrimers. Where the delivery vehicle is a liposome, the liposome can be any one of MLV (multilamellar vesicles), SUV (small unilamellar vesicles) and LUV (large unilamellar vesicles).

To facilitate delivery of a delivery vehicle to skin, tissue, tumor or cells of interest and/or to desirable subcellular locations within cells of interest, in some embodiments, the delivery vehicle contains one or more skin, tissue tumor or cellular and/or subcellular targeting signals. Many such targeting signals are known in the art. In some embodiments, the delivery vehicle contains a targeting signal, which targets the delivery vehicle to one or more of cancer cells and immune cells. For example, targeted delivery to immune cells can be achieved by using a delivery vehicle in the form of an antigen presenting liposome, virosome, bacterial phage-like carrier, micelles, etc. The delivery vehicle may also contain other modifications that facilitate delivery of the delivery vehicle and/or the encapsulated substance to cells of interest. In some embodiments, the delivery vehicle contains one or more of pegylation and connection to dendritic polymers. Pegylation can facilitate delivery of delivery vehicles to cells of interest by allowing the delivery vehicle to be maintained in the body for extended periods of time, e.g., by allowing the delivery vehicle to avoid the body's clearance systems. For example, pegylation increases of between 4 and 10% can increase the body's retention of the delivery vehicle from 200 to 1000 minutes. As described above, the delivery vehicle may be brought into contact with cells in any acceptable manner known in the art. In some embodiments, the delivery vehicle is brought into contact with cells through topical application or injection inside the skin, tissue or tumor. Topical application of a delivery vehicle results in an initial requirement that the delivery vehicle be able to permeate the skin, tissue or tumor's surface. In addition to the use of a plasma source, permeation can be further enhanced by modification of the delivery vehicle and/or the context in which the delivery vehicle is topically applied to the skin surface. For example, in some embodiments, the delivery vehicle is topically applied to the skin, tissue or tumor surface as part of a liquid, gel, or patch. Application of the delivery vehicle in this manner is particularly suitable for treating systemic conditions (e.g., leukemia or organ cancers) as the combination of the delivery vehicle and the liquid, gel, or patch can facilitate delivery of the delivery vehicle and the encapsulated substance to the bloodstream. In some embodiments, the delivery vehicle is suspended in a carrier vehicle. To aide delivery of a delivery vehicle and its contents to cells of interest, the carrier vehicle may contain at least one of a permeation enhancer, surfactant, detergent, anti-agglomeration agent, a gel, an oil, and a lipophilic substance.

Various known substances may be delivered to cells of interest through the use of a delivery vehicle in conjunction with a plasma source in the methods disclosed herein. Exemplary, non-limiting encapsulated substances include growth factors, polynucleotides, oligonucleotides, peptides, vaccines, DNA-based vaccines, protein-based vaccines, self-assembling 3D vaccines, nanoparticles, drugs, and cosmeceuticals. Encapsulation of these and other substances in a delivery vehicle can facilitate delivery to cells of interest by not only improving penetration through barriers present in the body, but also by providing a barrier such that cells other than cells of interest undergo limited exposure to the encapsulated substance. This is particularly desirable where the encapsulated substance is toxic to one or more cells types. This will often be the case where the encapsulated substance is a substance targeted towards killing cells, e.g., where the encapsulated substance is an anti-cancer agent. Multiple anti-cancer agents are known in the art and can be targeted for delivery to cells of interest using the methods described herein. Exemplary, non-limiting anti-cancer agents which can be delivered to cells of interest using the methods disclosed herein include Paclitaxel, Doxorubicin, Daunorubicin and Camptothecin.

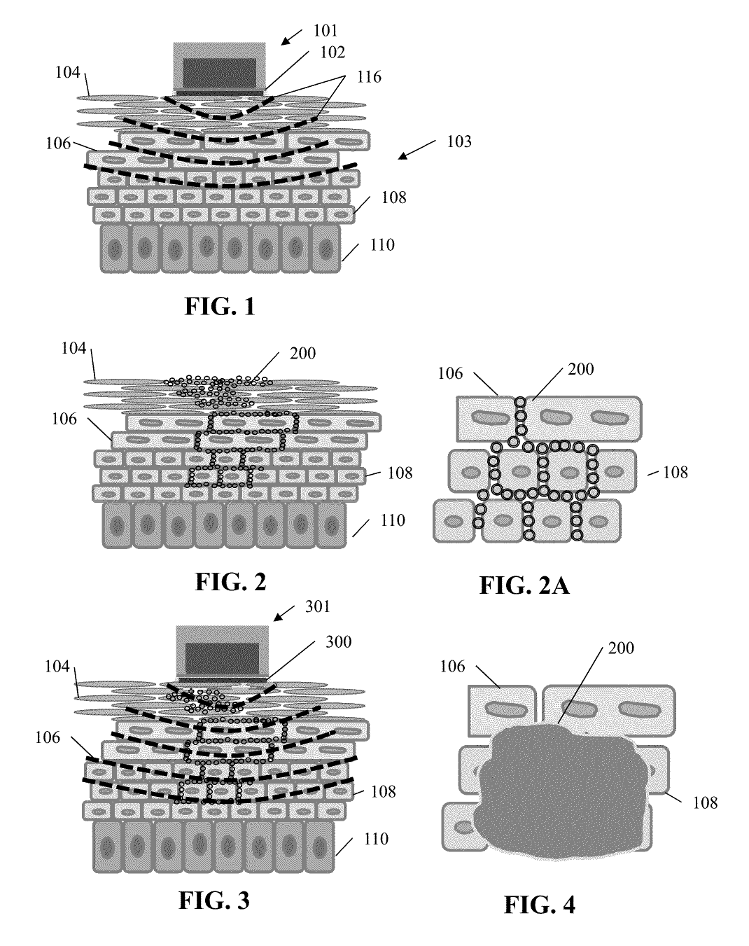

FIGS. 1-8 are exemplary illustrations of one or more portions of methods for intercellular and intracellular transport of delivery vehicles and breakdown of the delivery vehicles to release their contents. These one or more portions of the methods may be combined with one another in many different combinations. FIG. 1 is an exemplary illustration of applying plasma 102 to skin 103 to porate the skin. In the exemplary embodiment, plasma 102 causes electric field 116 to penetrate the layers of the skin 103 (stratum corneum 104, viable epidermis 106, dermis 108 and subcutaneous tissue 110). The plasma generator 101 is a direct barrier discharge (DBD) plasma generator. The DBD generators operate with a voltage source that has one or more polarity changes, or is pulsed. The applied voltage may be pulsed from 0 volts to the high voltage setting, or may be pulsed between a first voltage and a second voltage. The settings for the plasma generator 101 that generates the plasma 102 determines the amount of plasmaporation and the depth of penetration of the electrical fields 116. In some embodiments, the settings on the plasma generator are a moderate voltage of between about 3-20 KV, with a moderate pulse duration of between about 1 microsecond and 1 millisecond.

FIGS. 2 and 2A are exemplary illustrations of delivery vehicle 200 being applied to the area of the skin 103 that was plasmaporated in FIG. 1. The pores were opened between cells and the delivery vehicles 200 were absorbed into the skin to selected areas between the cells (Intercellular). In some embodiments, the methods illustrated by FIGS. 1 and 2 are replaced by injecting the delivery vehicle with one or more needles or other methods used to place the delivery in targeted areas between the cells.

FIG. 3 is an illustration of an exemplary method of breaking down the delivery vehicle to release the contents of the delivery vehicle in selected intercellular locations. In some exemplary embodiments, the plasma generator 301, which is a DBD plasma generator, is set at a high voltage, such as for example 30 kV, with a short duration, for example between 1 and 500 nanoseconds (ns), with a fast rise time of between about 3 and 5 kV/ns to cause the delivery vehicles 200, such as liposomes, to release the contents 200 within the skin as shown in FIG. 4.

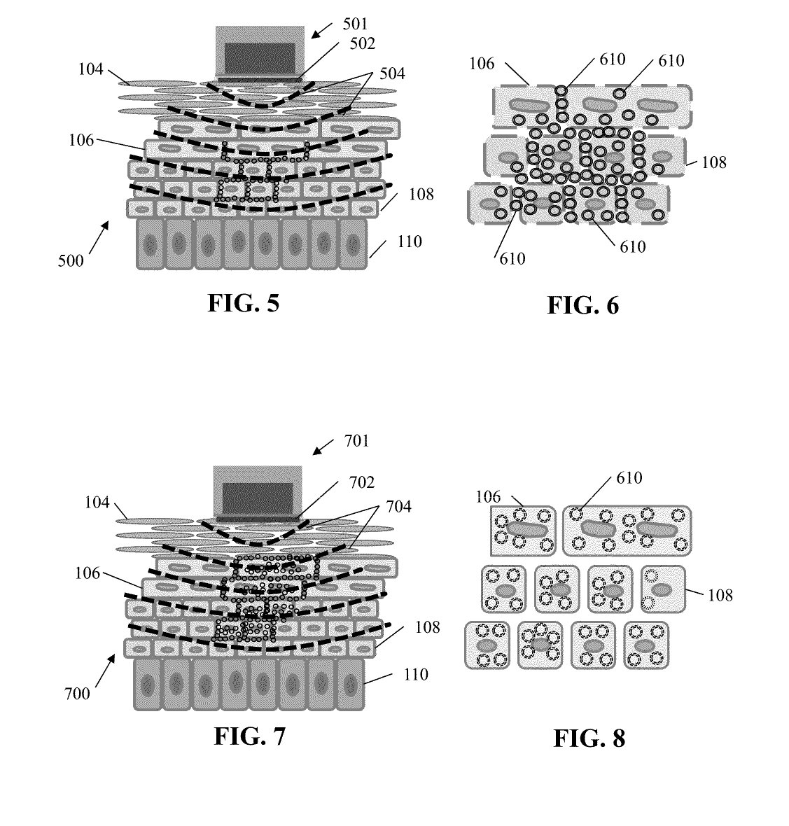

FIG. 5 illustrates an exemplary method of causing poration of the cells and cellular uptake of the delivery vehicles 610 into one or more cells 106, 108. Delivery vehicles 610 may be introduced intercellularly by any of the methods described above including through plasma poration or by injection into the targeted area. In some embodiments, the cellular uptake is caused by applying non-thermal plasma 502 to the selected area, wherein the plasma 502 was generated with a plasma generator 501 having a higher voltage, of between about 20- and 30 kV, with a short pulse duration of between about 10 ns and 1 μs. FIG. 6 is an exemplary illustration showing delivery vehicles 610 located both intercellularly and intracellularly.

FIG. 7 is an exemplary illustration of causing the delivery vehicles 610 to release their content intracellularly. The delivery vehicles 610 may be delivered into the cells 106, 108 by any means, including applying plasma to delivery vehicles that are located intracellularly and by allowing the cells 106, 108 to uptake delivery vehicles 610 on their own. The delivery vehicles 610 may be caused to release their contents inside the cells 106, 108 as shown in FIG. 8, by applying plasma 702 to the treated area to porate the delivery vehicle 106. In some embodiments, plasma 702 is generated by setting the plasma generator to a high voltage, of greater than about 30 kV for a short pulse duration of between about 100 picoseconds and 10 nanoseconds at a fast rise time of between about 3 and 5 kV/ns. FIG. 8 is an exemplary illustration of delivery vehicles 610 porated and having released their contents within cells 106, 108.

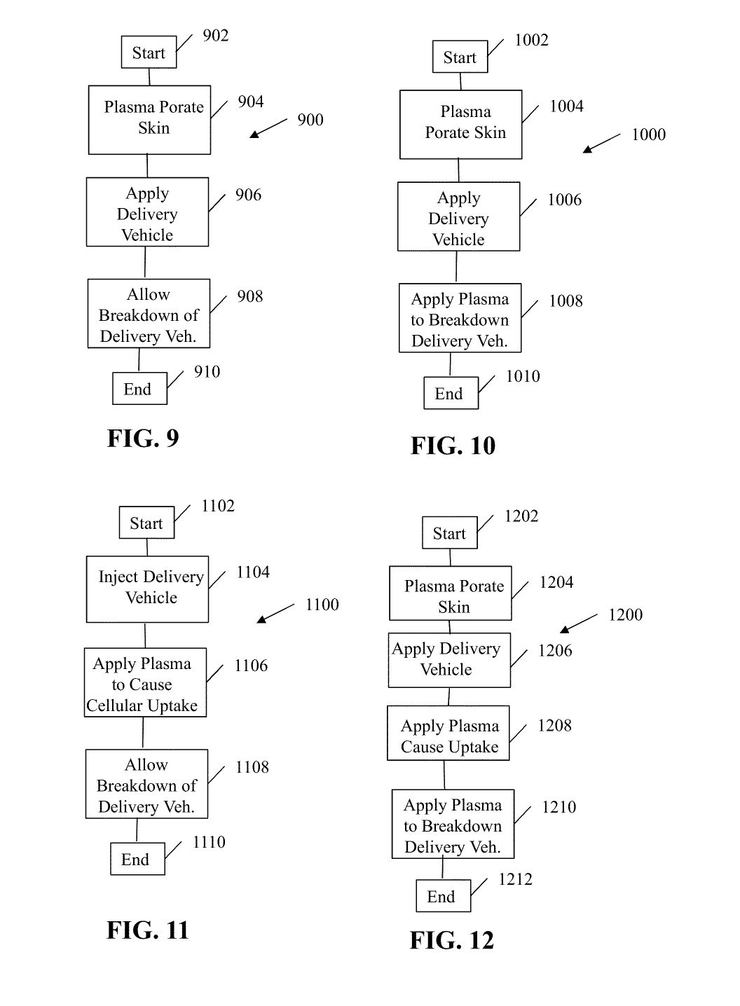

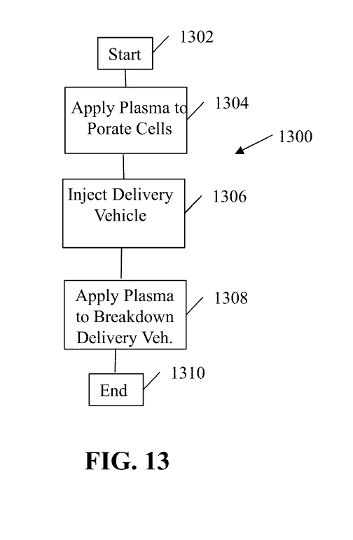

FIGS. 9-13 are exemplary methodologies of intercellular and intracellular delivery of substances encapsulated in delivery vehicles. Although the methods are described in a particular order, the steps may be performed in different orders. In addition, steps of the exemplary methodologies may be combined with other of the exemplary methodologies. In addition, steps may be added or removed from the exemplary methodologies.

The exemplary methodology 900 begins at block 902. At block 904, the skin is plasma-porated. The skin may be porated using plasma generated from a DBD plasma generator set at a moderate voltage of between about 3 and about 10 kV at a moderate pulse duration, of between about 1 microsecond and about 1 millisecond. At block 906, the delivery vehicle is topically applied to the surface of the skin. In some embodiments, the delivery vehicles are liposomes that encapsulate a substance. The substance may be, for example, drugs, vaccines, cosmetics, DNA, RNA, growth factors, or the like. After a period of time, such as, for example, between about 1 and 60 minutes, the delivery vehicles travel to the desired depth (which in some embodiments, may be controlled by the plasma generator settings). The delivery vehicles are allowed to break down at block 908 and deliver their contents. The methodology ends at block 910.

Another exemplary methodology 1000 begins at block 1002. At block 1004, the skin is plasma-porated. The skin may be porated using plasma generated from a DBD plasma generator set at a moderate voltage of between about 3 and about 10 kV at a moderate pulse duration, of between about 1 microsecond and about 1 millisecond. At block 1006, the delivery vehicles are topically applied to the surface of the skin. In some embodiments, the delivery vehicles are liposomes that encapsulate a substance. The substance may be, for example, drugs, vaccines, cosmetics, DNA, RNA, growth factors, or the like. After a period of time, such as, for example, between about 1 and 60 minutes, the delivery vehicles travel to the desired depth (which in some embodiments, may be controlled by the plasma generator settings). In some embodiments, it is desired to wait at least 10 minutes to allow the delivery vehicles to travel to the desired depth. In some embodiments an additional step of wiping or scrubbing the treated area of skin to remove any delivery vehicles or drugs that have not traveled into the skin. This may be desirable to prevent interaction with exposed liposomes or their contents with plasma. Contacting exposed liposomes with plasma may cause the liposome to release its content and also destroy or modify the drug, DNA or other contents of the exposed liposome.

At block 1008, plasma is applied to the targeted area to porate or breakdown the delivery vehicle to cause the delivery vehicle to release their contents between the cells (intercellularly). In some embodiments, the plasma is generated using a high voltage of greater than about 30 kV, with short pulse duration of between about 1 and about 500 ns at a fast rise time of about 3 to about 5 kV/ns to cause the release of the contents. In some embodiments, blocks 1004 and 1006 may be eliminated by injecting the delivery vehicles into the skin with, for example, one or more needles. The methodology ends at block 1010.

Another exemplary methodology 1100 begins at block 1102. At block 1104, delivery vehicles are injected within the skin. In some embodiments, the delivery vehicles are liposomes that encapsulate a substance. The substance may be, for example, drugs, vaccines, cosmetics, DNA, RNA, growth factors, or the like. Optionally, block 1104 may be replaced by one or more of the blocks identified above for transporting delivery vehicles to selected intercellular areas. At block 1106, plasma is applied to the selected area to porate the cells and cause cellular uptake of the delivery vehicle. In some embodiments, the plasma is generated by setting the plasma generator to a higher voltage of between about 10 and about 30 kV at a short pulse duration of between about 10 ns and 1 μs to achieve intracellular uptake. The delivery vehicle is allowed to breakdown and release the contents at block 1108 and the exemplary methodology ends at block 1110.

Another exemplary methodology 1200 begins at block 1202. At block 1204, the skin is plasma-porated. The skin may be porated using plasma generated from a plasma generator set at a moderate voltage of between about 3 and about 10 kV at a moderate pulse duration, of between about 1 microsecond and about 1 millisecond. At block 1206, the delivery vehicles is topically applied to the surface of the skin. In some embodiments, the delivery vehicles are liposomes that encapsulate a substance. The substance may be, for example, drugs, vaccines, cosmetics, DNA, RNA, growth factors, or the like. At block 1208, plasma is applied to the selected area to porate the cells and cause cellular uptake of the delivery vehicle. In some embodiments, the plasma is generated by setting the plasma generator to a higher voltage of between about 10 and about 30 kV at a short pulse duration of between about 10 ns and 1 μs to achieve intracellular uptake. At block 1210, plasma is applied to porate or breakdown the delivery vehicle to cause the delivery vehicle to release their contents between the cells (intercellularly). In some embodiments, the plasma is generated using a high voltage of greater than about 30 kV, with a short pulse duration of between about 1 and about 500 ns at a fast rise time of about 3 to about 5 kV/ns to cause the release of the contents. In some embodiments, blocks 1204 and 1206 may be eliminated by injecting the delivery vehicles into the skin with, for example, one or more needles. The methodology ends at block 1212.

Another exemplary methodology 1300 begins at block 1302. At block 1304, plasma is applied to the selected area to porate the cells so they will uptake the delivery vehicle. In some embodiments, the plasma is generated by setting the plasma generator to a higher voltage of between about 10 and about 30 kV at a short pulse duration of between about 10 ns and 1 is to achieve intracellular uptake. At block 1306, the delivery vehicle is injected into the targeted area, and is taken up by the porated cells. In some embodiments, the delivery vehicles are liposomes that encapsulate a substance. The substance may be, for example, drugs, vaccines, cosmetics, DNA, RNA, growth factors, or the like. At block 1308, plasma is applied to porate or breakdown the delivery vehicle to cause the delivery vehicle to release their contents between the cells (intercellularly). In some embodiments, the plasma is generated using a high voltage of greater than about 30 kV, with a short pulse duration of between about 1 and about 500 ns at a fast rise time of about 3 to about 5 kV/ns to cause the release of the contents. In some embodiments, blocks 1304 and 1306 may be eliminated by injecting the delivery vehicles into the skin with, for example, one or more needles. The methodology ends at block 1310.

### EXAMPLE

The following example illustrates specific and exemplary embodiments, features, or both, of the methods disclosed herein. The example is provided solely for the purpose of illustration and should not be construed as limitations on the present disclosure.

### Example 1

### Delivery Depths Acieved with Plasmaporation and Electroporation

To compare the ability of plasmaporation to facilitate permeation of delivery vehicles and their encapsulated substances to cells of interest in skin, tissue or tumor, 100 nm commercially available DOPC/CHOL/mPEG-DSPE Liposomes labeled with Fluorescein DHPE (Formumax, Palo Alto, Calif.) were applied topically to porcine skin after electroporating or plasmaporating the skin. Briefly, electroporation treatment was carried out using the Harvard Apparatus BTX810 at a setting of 100-1000 V/cm, using ten 100 microsecond −100 millisecond pulses allowing 100 milliseconds between pulses. Plasmaporation treatment was carried out using an Eagle Harbor Technologies, Seattle NSP-1000 nanosecond pulsed plasma at a setting of 20 kV, using continuous pulses 60-300 nanosecond in duration at a frequency of 200-500 Hz over a period of 30 seconds or 5-10 distinct pulses of 60-500 nanoseconds in duration. The skin was imaged non-invasively one hour after liposome application via confocal imaging using Vivascope® 1500 Multilaser Skin imaging system. Specific settings used with tested samples are shown in table 1, below.

TABLE 1
Electroporation and plasmaporation settings used with tested samples.

| | | Applied | Pulse | Pulse Interval | | Time |
|---|---|---|---|---|---|---|
| Sample | Type | Voltage | Duration | or Frequency | # pulses | (s) |
| Control | | — | — | — | — | — |
| Sample 1 | Electroporation | 100 V/cm | 100 us | 100 ms | 10 | — |
| Sample 2 | Electroporation | 100 V/cm | 100 ms | 100 ms | 10 | — |
| Sample 3 | Electroporation | 200 V/cm | 100 us | 100 ms | 10 | — |
| Sample 4 | Electroporation | 200 V/cm | 100 ms | 100 ms | 10 | — |
| Sample 5 | Electroporation | 1000 V/cm | 300 us | 100 ms | 10 | — |
| Sample 6 | Plasmaporation | 20 kV | 60 ns | — | 5 (PDP) | — |
| Sample 7 | Plasmaporation | 20 kV | 300 ns | — | 5 (PDP) | — |
| Sample 8 | Plasmaporation | 20 kV | 60 ns | 200 Hz | — | 30 (PDP) |
| Sample 9 | Plasmaporation | 20 kV | 60 ns | 500 Hz | | 30 (PDP) |
| Sample 10 | Plasmaporation | 20 kV | 300 ns | 200 Hz | — | 30 (PDP) |
| Sample 11 | Plasmaporation | 20 kV | 500 ns | — | 10 (PD) | — |

(Legend - PD: Plasma application followed by topical application of liposomes (delivery vehicle); PDP: Plasma application followed by topical application of liposomes followed by another plasma application).

As shown in table 1, eleven samples (including a control not subjected to either plasmaporation or electroporation) were tested under various combinations of parameters. Briefly, table 1 provides the sample number, the type of exposure, applied voltage of the pulses, pulse duration, interval between pulses or frequency of the pulses, number of total pulses applied, and the total time over which pulses were applied.

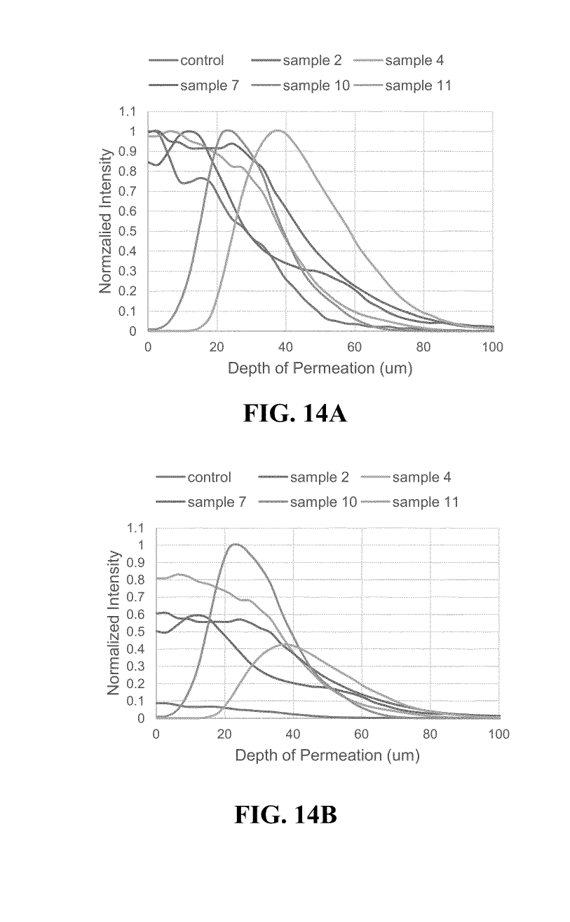

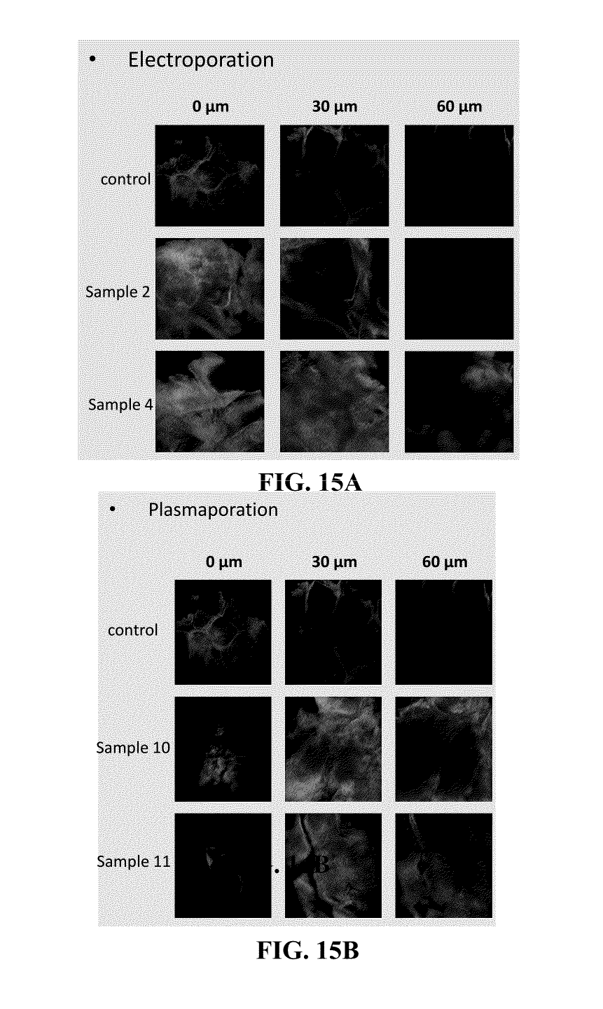

The results for representative samples from table 1 are shown in FIGS. 14A-14B and 15A-15B. FIG. 14A shows the intensity detected at a given depth normalized for the maximum intensity for an individual sample. As shown in FIG. 14 A, in contrast to electroporation samples 2 and 4, and the control, where the maximum intensity was detected at or below 20 micrometers, plasmaporation samples 10 and 11 demonstrated maximum intensities at depths between 20 and 40 micrometers. Thus, the maximum depth to which the delivery vehicle can be delivered is greater with plasmaporation than with electroporation. FIG. 14B shows the intensity detected at a given depth normalized for the maximum intensity across all samples. As shown in FIG. 14B, in contrast to the electroporation samples 2 and 4, and the control, where the majority of the intensity is below 20 micrometers in depth, plasmaporation samples 10 and 11 show the majority of the intensity above 20 micrometers in depth. In fact, for plasmaporation sample 11, greater than 40% of the intensity occurred at almost 40 micrometers in depth. Thus, the average depth to which a delivery vehicle is delivered is greater under plasmaporation than under electroporation. FIGS. 15A and 15B show the raw visualization of the intensity detected under electroporation (FIG. 15A) and plasmaporation (FIG. 15B) treatments. As shown in FIG. 15A, most of the intensity for the control and electroporation samples is detected at the surface. In fact, only electroporation sample 4 provides some delivery of the delivery vehicle to a depth of 60 micrometers. By contrast, as shown in FIG. 15B, the majority of the intensity for plasmaporation samples 10 and 11 is seen below the skin surface with a significant amount of the delivery vehicle being delivered to a depth of 60 micrometers. Over the samples where delivery of the delivery vehicle was visualized, plasmaporation delivered the delivery vehicle to an average depth of 60 micrometers while electroporation delivered the delivery vehicle to an average depth of 30 micrometers. Furthermore, a greater percentage of all delivery vehicle was delivered into the skin with plasmaporation than with electroporation.

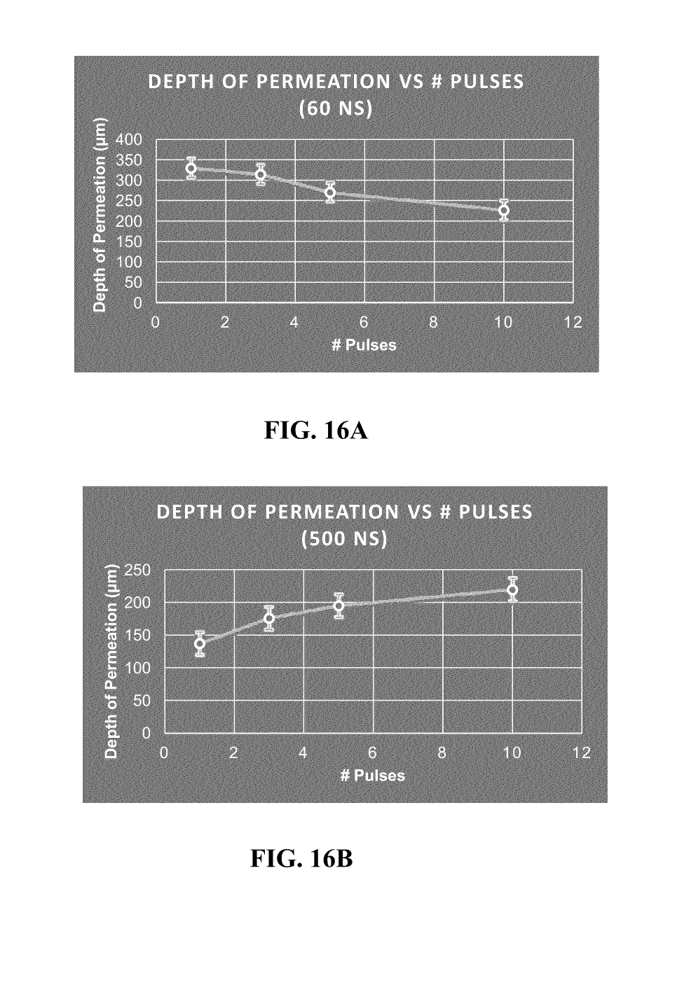

It was determined that depth of permeation depends on various plasma parameters that include applied voltage, pulse duration, frequency, and number of applied pulses. Nanosecond pulsed plasma is able to deliver 100 nm diameter liposomes transdermally within 1 hour of treatment. The graphical representations in FIGS. 16A-19 were obtained with the following setups. Ex vivo porcine skin was treated with nanosecond pulsed plasma by varying different plasma treatment parameters including pulse duration, frequency, voltage, number of applied pulses, time of plasma treatment and mode of plasma operation (continuous or pulsed). Immediately after plasma treatment 100 nm diameter liposomes were applied topically to the treated area. After 1 hour, 10 mm punch biopsies were obtained and immediately preserved in 10% neutral buffered formalin. Biopsies were processed using standard histology processes and then imaged under a fluorescently enabled microscope. Depth of permeation was determined from obtained images.

FIG. 16A is a graphical representation of the depth of permeation of 100 nm diameter liposomes based on the number of pulses. Each pulse duration was 60 ns. Surprisingly, it was observed that at short pulse durations, the liposomes penetrated deeper with less pulses, i.e. 1 60 ns pulse caused the liposomes to penetrate to a depth of about 325 μm while 10 60 ns pulses caused penetration to a depth of about 200 μm). However, when the pulse duration was increased to 500 ns, as shown in FIG. 16B, 1 500 ns pulse resulted in a lower depth of permeation (a depth of about 130 μm) than 10 500 ns pulses (a depth of about 220 μm).

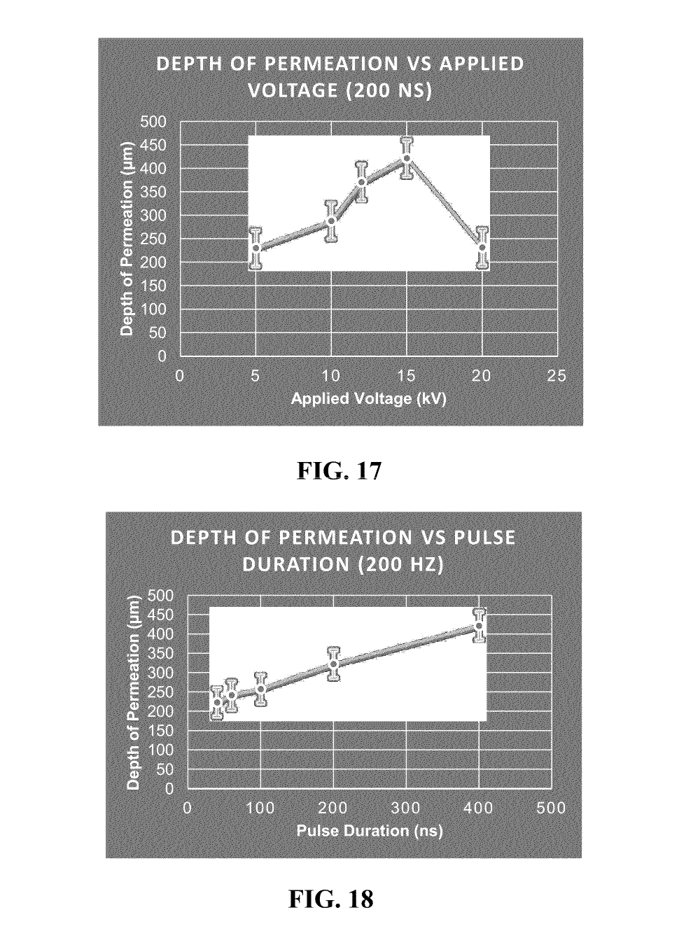

FIG. 17 is a graphical representation of the depth of permeation of plasma in a pulsed mode of treatment based on the applied voltage at 200 ns pulse duration and 5 pulses. The experiments demonstrate that the permeation increases as the voltage increases up to a point (e.g. about 15 kV) and then the depth of permeation decreases as the voltage increases.

FIG. 18 illustrates depth of permeation in a continuous mode of operation versus pulse duration at an operating frequency of 200 Hz. As the pulse duration increases with the frequency (200 Hz), applied voltage (20 kV) and the time of treatment (30 s) being fixed, the depth of permeation increases.

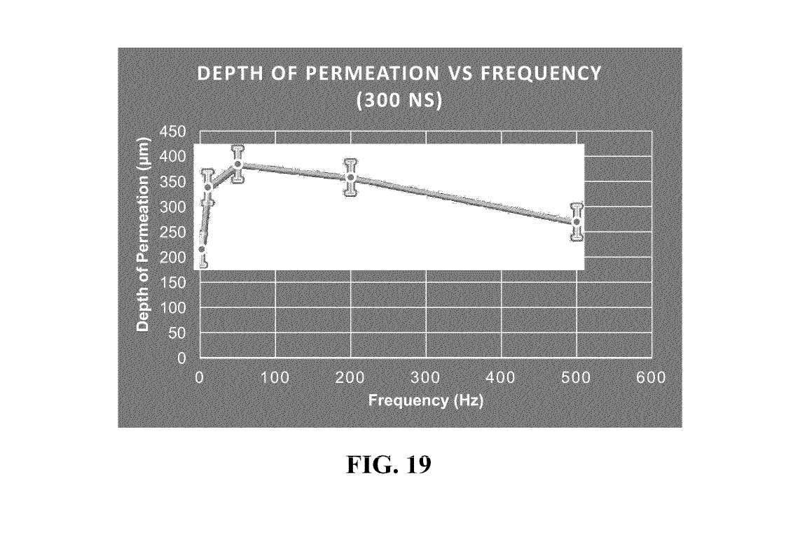

FIG. 19 illustrates depth of permeation in a continuous mode of operation versus frequency at a pulse duration of 300 ns and an applied voltage of 20 kV. The experiments demonstrate that the permeation increases as the frequency increases up to a point (e.g. about 50 Hz) and then the depth of permeation decreases as the frequency increases.

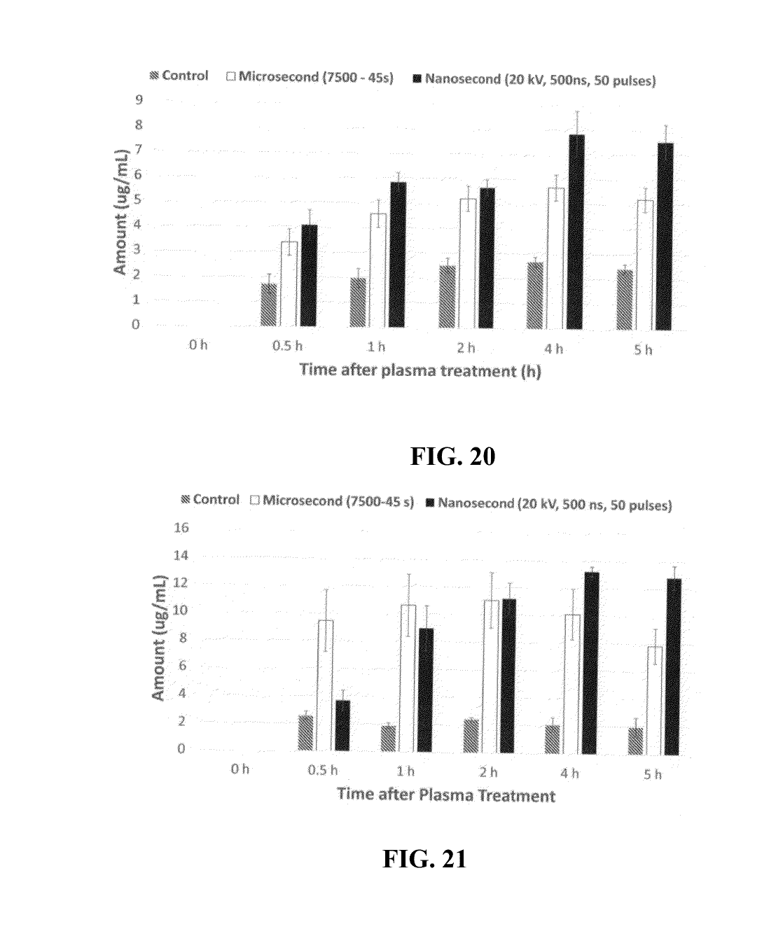

FIGS. 20 and 21 demonstrate the viability of using plasma to cause a vehicle carrier to release its contents in a desired location either intercellularly or intracellularly. In this exemplary experiment, porcine skin taken from the abdomen with intact stratum corneum was used. The skin was kept at -80° C. until the day of treatment. On the day of treatment the skin was thawed to room temperature and kept in a humidified box for 1 hour. The skin was washed with water and pat-dried with paper towels. The skin was cut into 1″×1″ pieces and was placed between the donor and receiver compartments in a Franz-Diffusion setup with temperature maintained at 32+/−1° C. The delivery vehicles selected and used for the experiments were liposomes encapsulating carboxyfluorescein (13 mM)−100 nm diameter (original concentration of 5 mg/ml)). The liposomes were formulated to contain 42.9 mg DOPC and 18.6 mg cholesterol and hydrated in 2.0 ml buffer (20 mM HEPES, pH 7.0, 10% sucrose). The liposomes were reconstituted in sterile deionized water to a working concentration of either 0.5 mg/mL or 2 mg/mL. Two 100 μl intradermal injections in to the skin were followed by a plasma treatment. One plasma treatment was conducted using a microsecond plasma power supply with settings at 7500-45 s (16 kV, 2500 Hz, 5 μs, 100% duty cycle, 45 s treatment). The second type of plasma treatment was conducted using a nanosecond plasma power supply with settings at 50 pulses (20 kV, 500 ns). During the experiment, the treated skin was mounted on a diffusion chamber. The receiver compartment was filled with 10 ml of water. 500 μL samples were collected from the receiver compartment at time intervals 0, .5, 1, 2, 4 and 5 hours. Fluorescence intensity (Ex/Em: 495 nm/525 nm) was measured using microplate reader. The intact liposomes had quenched fluorescence due to the high concentration of fluorescent molecules that result in more close-range interactions and subsequently lower intensity. The porated liposomes, or liposomes that released their contents, exhibited stronger fluorescence due to the lower concentration of fluorescent molecules that were released into the area around the porated liposome that result in less close-range interactions and subsequently higher intensity.

FIG. 20 illustrates results for 0.5 mg/ml of liposome having carboxyfluorescein injected into the skin. As can be seen, the use of either plasma treatment was significantly better at each time interval from 0.5 hours through 5 hours with respect to the control and the nanosecond plasma treatment content release result were superior to the microsecond plasma treatment results. The results demonstrate that both plasma treatments were able to cause the liposomes to release their contents within the skin.

FIG. 20 illustrates results for 2 mg/ml of liposome having carboxyfluorescein injected into the skin. As can be seen, the samples using plasma treatment were significantly better at 0.5 hours through 5 hours with respect to the control and the nanosecond plasma treatment results were superior to the microsecond plasma treatment results with the longer time periods, however the microsecond plasma treatment appears to be cause a higher content release in shorter time. Again, the results demonstrate that both plasma treatments were able to cause the liposomes to release their contents within the skin.

Applicants also conducted a number of experiments related to contacting the liposomes dissolved in water with plasma to assess the ability of the liposomes to withstand the treatment to protect the encapsulated drug when contacted by plasma. Applicants discovered that treating liposomes dissolved in water with the microsecond pulsed plasma treatments (settings at 16 kV, 5 μs, 2500 Hz, 45s) resulted in damage to both the lipososmes and to the encapsulated substance. Treatment of the liposomes dissolved in water with nanosecond pulsed plasma (settings at 20 kV, 500 ns, 50 pulses) resulted in premature release of the contents of the liposomes, but did not appear to damage the encapsulated drug. Accordingly, it is not recommended to directly contact liposomes with plasma prior to the liposomes being located within the skin, as encapsulating drugs in liposomes does not make them amenable to be interacted with direct plasma and/or prematurely releases the contents.

Unless otherwise indicated herein, all sub-embodiments and optional embodiments are respective sub-embodiments and optional embodiments to all embodiments described herein. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions or formulations, and illustrative examples shown and described.

Accordingly, departures may be made from such details without departing from the spirit or scope of the applicant's general disclosure herein.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

As used in the description of the invention and the appended claims, the singular forms “a,” “an,” and “the” are intended to include the plural forms as well, unless the context clearly indicates otherwise.

We claim:

1. A method of improving delivery of a substance encapsulated in a delivery vehicle to cells of interest in skin, tissue or tumor, the method comprising:
(a) bringing a delivery vehicle encapsulating a substance in contact with cells; and
(b) exposing skin, tissue, tumor or cells to a plasma source to cause the delivery vehicle to release the substance.

2. The method of claim 1, comprising:
(a) exposing the skin, tissue or tumor to a plasma source under conditions which temporarily porate the skin; and
(b) topically applying the delivery vehicle to the porated skin to bring the delivery vehicle encapsulating a substance in contact with the cells.

3. The method of claim 1, comprising injecting the delivery vehicle inside the skin, tissue or tumor to bring the delivery vehicle encapsulating a substance in contact with the cells.

4. The method of claim 1, comprising:
(a) injecting the delivery vehicle inside the skin, tissue or tumor to bring the delivery vehicle in contact with cells inside the skin, tissue or tumor; and
(b) exposing the skin, tissue or tumor to a plasma source under conditions which temporarily porate the cells inside the skin, tissue or tumor.

5. The method of claim 1, further comprising exposing the skin, tissue or tumor to a plasma source under conditions which porate cells in skin, tissue or tumor and injecting the delivery vehicle inside the skin.

6. The method of any one of claims 1, wherein the delivery vehicle is a liposome, an artificial virosome, a bacterial phage like carrier, hollow nanoparticles, or a micelle.

7. The method of any one of claims 1, wherein the encapsulated substance comprises at least one of growth factors, polynucleotides, oligonucleotides, peptides, small RNAs, DNA based vaccines, protein based vaccines, vaccines, nanoparticles, drugs, self-assembling 3D vaccines and cosmetics.

8. The method of any one of claims 1, wherein the delivery vehicle contains a cellular targeting signal or tag.

9. The method of claim 8, wherein the cellular targeting signal targets the delivery vehicle to one or more of cancer cells and immune cells.

10. The method of claim 9, wherein the encapsulated substance comprises an anti-cancer agent.

11. The method of claim 2, wherein the delivery vehicle is topically applied to the skin as part of a liquid, gel, or patch.

12. The method of claim 2, wherein the delivery vehicle comprises one or more of pegylation and connection to dendritic polymers.

13. The method of any one of claims 1, wherein the delivery vehicle is suspended in a carrier vehicle.

14. The method of claim 13, wherein the carrier vehicle comprises at least one of a permeation enhancer, surfactant, detergent, anti-agglomeration agent, a gel, an oil, and a lipophilic substance.

15. A method of delivering a substance encapsulated in a delivery vehicle to inside cells of interest, the method comprising:
applying plasma to skin to porate the skin;
topically applying a vehicle carrier to the skin;
applying plasma to porate cells in the skin; and
applying plasma to cause the vehicle carrier to release the contents of the vehicle carrier.

16. The method of claim 15 wherein the plasma is applied to porate the cells prior to applying the vehicle carrier to the skin.

17. The method of claim 15 wherein the plasma is applied to porate the cells after applying the vehicle carrier to the skin.

18. A method of delivering a substance encapsulated in a delivery vehicle to inside cells of interest, the method comprising:
injecting a vehicle carrier in skin;
applying plasma to porate cells in the skin; and
applying plasma to cause the vehicle carrier to release the contents of the vehicle carrier.

19. The method of claim 18 wherein applying the plasma to porate the cells occurs prior to injecting the vehicle carrier in the skin.

20. The method of claim 18 wherein applying the plasma to porate the cells occurs after injecting the vehicle carrier in the skin.

21. A method of delivering a substance encapsulated in a delivery vehicle to a selected depth in the skin:
applying plasma to skin to porate the skin;
wherein the plasma settings are selected to deliver the delivery vehicle to a selected depth.

22. The method of claim 21 wherein the selected depth is between about 20 μm and about 200 μm.
